(19) United States
(12) Patent Application Publication
(10) Pub. No.: US 2025/0345272 A1
(43) Pub. Date: Nov. 13, 2025

(54) INJECTABLE AND 3D EXTRUSION PRINTABLE HYDROPHILIC SILICONE-BASED HYDROGEL FOR CONTROLLED DRUG RELEASE

(71) Applicants: Xiaowu Shirley TANG, Waterloo (CA); Centre for Eye and Vision Research Limited, Hong Kong (HK)

(72) Inventors: Xiaowu Shirley TANG, Waterloo (CA); Sayan GANGULY, Waterloo (CA)

(21) Appl. No.: 19/201,972

(22) Filed: May 8, 2025

Related U.S. Application Data
(60) Provisional application No. 63/644,461, filed on May 8, 2024.

(51) Int. Cl. A61K 9/06; A61K 31/43; A61K 47/32; A61K 47/34; A61K 47/38; B33Y 10/00; B33Y 70/10; C09D 11/101; C09D 11/102; C09D 11/107; C09D 11/14

(52) U.S. Cl. CPC A61K 9/06; A61K 31/43; A61K 47/32; A61K 47/34; A61K 47/38; B33Y 10/00; B33Y 70/10; C09D 11/101; C09D 11/102; C09D 11/107; C09D 11/14

(57) ABSTRACT

This invention arrests the hydrophilic silicone macrochains into semi-interpenetrating polymer network via in situ photo-gelation assisted 3D microextrusion printing technique. The printed hybrid hydrogel has shown microporous morphology with tunable diffusion behaviour. The flow behaviour of the hydrogel has been tested showing high elastic modulus, low tan δ, high gel strength, and delayed network rupturing behaviour. The uniaxial compression test showed the almost zero permanent set which could promote it as an elastomer mimetic soft biomaterial. Moreover, the drug loading into the hydrogel has been performed which showed hydrophilic silicone dependent non-Fickian anomalous transport. The encapsulated drug stability inside hydrogel matrices also showed no deterioration of the pristine drug molecules even after one month storage. This could be the first hydrodrophilic silicone based soft biomaterial will serve as an excellent controlled drug delivery device.

### CROSS-REFERENCE TO RELATED APPLICATIONS

The present application claims the priorities from the U.S. provisional patent application Ser. No. 63/644,461 filed May 8, 2024, and the disclosure of which is incorporated herein by reference in its entirety.

### FIELD OF THE INVENTION

The present invention generally relates to the field of material science and additive manufacturing. More specifically, the present invention relates a composition and process of silicone-based hydrogel for ophthalmic drug delivery with stimuli responsiveness.

### BACKGROUND OF THE INVENTION

Over the past decade, three-dimensional (3D) printing has become a key technology in manufacturing, used across industries like automotive, aerospace, dentistry, soft robotics, and pharmaceuticals. In healthcare, it's rapidly advancing, enabling complex structures like skeletal scaffolds and hydrogel-based heart models. This method shows great potential in bioengineering, drug delivery, and medical devices.

There are various 3D printing methods, with inkjet, laser-assisted, and extrusion-based printing being the most common. Among them, extrusion-based printing, or direct ink writing (DIW), is widely used and allows hydrogels to be used as printing materials. Microextrusion-based (ME) 3D printing has gained greater popularity than other types of 3D printing because of its versatility and the flexibility to use a wide variety of inks with varying viscosities. The ink must be fluid enough to be extruded via a nozzle, yet solid enough to stay there on the support substrate once it's been printed. Maintaining shape accuracy in ME is difficult because of competing ink requirements, which are based on ink flow characteristics that address gravitational pull and surface energyl,2. Without a speedy fixing procedure, printed objects might collapse under the weight of gravity once they reach a particular height, which is in turn regulated by the viscoelastic qualities of the ink used in the printing process.

For extrusion-based 3D printing, the ink must meet several key criteria: it should have adequate viscosity to stay in the injector, flow easily under pressure during extrusion, maintain shape fidelity by retaining viscosity when in contact with the printing platform, and fix quickly to prevent the printed structure from collapsing. The combination of microextrusion printing and photocuring was first reported to print gelatin methacryloyl based hydrogel3. Gelatin and alginated based blend hydrogel were reported via ME printing method for scaffold fabrication4. These hydrogels were used for tissue engineering applications and sustained/controlled release platforms of therapeutic payloads.

Recent advancements in drug delivery systems using hydrogels have been significant, as reflected in the extensive body of published research. In particular, ocular drug delivery via hydrogels, such as drug-eluting contact lenses (CLs), has garnered increasing interest. These hydrogel-based devices are especially promising for delivering topical ophthalmic treatments. As of the contemporary report, CLs can increase drug bioavailability in the eye by at least 50% compared to conventional eye-drops suspensions (1-5%) 5. However, choosing an appropriate CLs material still is a big challenge where wetting and eye comfort of the patient are the most prioritized section. Hydrogel-based systems were once the most promising materials for CLs due to their higher water content, durability, and biocompatibility. However, their limited oxygen permeability hindered broader use. This issue was addressed by silicone materials, which are more elastic and oxygen-permeable, reducing hypoxia-related issues. However, silicone-based CLs can be less comfortable for sensitive eyes due to eye deposits and silicone intolerance. Moreover, when incorporating drug-eluting properties into contact lenses, silicone-based systems face significant drawbacks compared to hydrogels. One potential solution is to combine both inorganic and organic polymeric systems to address these challenges. For drug-eluting contact lenses, a key limitation is drug encapsulation, where the drug molecules are trapped within the gel matrices and released in a controlled, delayed manner.

Therefore, there is a need in the art for an improved material system for drug-eluting contact lenses that combines the best properties of both hydrogels and silicones.

### SUMMARY OF THE INVENTION

The present invention addresses long-standing challenges in the field of ocular drug delivery, particularly the difficulty in achieving sustained release from contact lens-compatible materials that also exhibit adequate oxygen permeability, mechanical stability, and patient comfort.

In one aspect, the present invention provides an injectable, extrusion-printable, and biocompatible formulation for 3D printing of a drug-releasing hydrogel. The formulation includes an aqueous dispersion of cellulose nanocrystals (CNCs) in an amount effective to impart shear-thinning behavior and yield stress sufficient to prevent nozzle dripping during pauses in extrusion, a monomer mixture comprising acrylamide (AM) and 2-hydroxyethyl methacrylate (HEMA), wherein the AM to HEMA weight ratio is between 1:3 and 3:1, a crosslinker, a photoinitiator, and an amine-functionalized silicone elastomer (AS). The formulation undergoes in situ ultraviolet-induced polymerization to form a semi-interpenetrating polymer network hydrogel with a mesh size of 50 Å to 150 Å. The hydrogel exhibits a tan delta (tan δ) value of less than 0.1 under oscillatory shear, indicating high elasticity.

In one embodiment, the AS content is in a range of 0.1 wt % to 10 wt %, and the CNCs content is in a range of 5 wt % to 15 wt %, based on total formulation weight.

In one embodiment, the photoinitiator includes lithium phenyl-2,4,6-trimethylbenzoylphosphinate (LAP), 2-hydroxy-4′-(2-hydroxyethoxy)-2-methylpropiophenone), 2,2′-Azobis [2-methyl-N-(2-hydroxyethyl) propionamide] or their combination thereof, or any water soluble photo-initiator. The % in the formulation will be different for different photoinitiator.

In one embodiment, the crosslinker includes N,N′-methylenebisacrylamide (MBA), or a combination thereof, or any water soluble divinylic acrylates. The % in the formulation will be different for different diacrylates.

In one embodiment, the formulation exhibits a shear-thinning behavior, characterized by a decrease in viscosity from 10⁴ Pa·s at a shear rate of 0.1 s⁻¹ to 0.1 Pa·s at a shear rate of 100 s⁻¹.

In one embodiment, the formulation displays a yield stress of approximately 1.8 Pa to 2.7 Pa when tested in compression mode.

In one embodiment, the semi-interpenetrating polymer network hydrogel exhibits zero permanent deformation following cyclic compression loading, a diffusion exponent (n) in the range of 0.5 to 0.75 indicative of non-Fickian anomalous transport behavior, and tunable oxygen permeability and mesh size modulated by the content of the AS.

In one embodiment, the semi-interpenetrating polymer network hydrogel has a Young's modulus greater than 10 kPa and an ultimate compressive strength of at least 50 kPa.

In one embodiment, the semi-interpenetrating polymer network hydrogel maintains dimensional and structural stability after immersion in phosphate-buffered saline at 37° C. for at least 10 days, and wherein the drug encapsulated therein retains at least 90% of its original chemical integrity and ultraviolet absorbance profile after storage for at least one month.

In one embodiment, the semi-interpenetrating polymer network hydrogel adheres to the periphery of a contact lens without delamination, distortion, or optical interference, and the semi-interpenetrating polymer network hydrogel conforms to the curvature of the cornea or sclera under ambient conditions.

In another aspect, the present invention provides a 3D printing method for fabricating a structurally stable semi-interpenetrating polymer network hydrogel. The method includes dispersing cellulose nanocrystals (CNCs) in water to form a shear-thinning base fluid; mixing the shear-thinning base fluid with a monomer mixture, a crosslinker, a photoinitiator, and an amine-functionalized silicone elastomer to form a precursor ink complex; extruding the ink complex into a predefined pattern using a microextrusion-based 3D printer, and subjecting extruded predefined pattern to continuous ultraviolet irradiation at a wavelength of 365 nm for a duration of 5 to 10 seconds to initiate polymerization and form the structurally stable semi-interpenetrating polymer network hydrogel. Simultaneous ultraviolet exposure and extrusion are employed to achieve in situ gelation during layer-by-layer deposition.

In one embodiment, the method further includes incorporating a therapeutic agent into the ink complex. The therapeutic agent is selected from antibiotics, anti-inflammatory drugs, anti-glaucoma drugs, or a combination thereof.

In one embodiment, the structurally stable semi-interpenetrating polymer network hydrogel sustains drug release for at least 8 hours post-application.

In one embodiment, the structurally stable semi-interpenetrating polymer network hydrogel exhibits no permanent deformation after undergoing at least 10 cycles of 20% strain.

In one embodiment, the structurally stable semi-interpenetrating polymer network hydrogel is printed in annular patterns with diameters of at least 10 mm. The minimum diameter is constrained by the printing resolution, typically around 10 mm, while the maximum is limited only by the printer's capacity, which can be quite large. In essence, this formulation and extrusion-based method allow users to print annular patterns with diameters determined by the capabilities of their specific printer.

In another aspect, the present invention provides a drug delivery system comprising the semi-interpenetrating polymer network hydrogel of claim 1 printed on a substrate.

In one embodiment, the substrate includes contact lenses, ocular patches, corneal bandages, or biodegradable ocular films.

In one embodiment, the semi-interpenetrating polymer network hydrogel has an oxygen permeability that exceeds that of poly (HEMA)-based hydrogel lenses by at least 25%.

In one embodiment, the semi-interpenetrating polymer network hydrogel is printed in a manner that allows the hydrogel to be detached and re-adhered without delaminating or compromising its structural integrity.

The developed hydrogel is tested by rheometry, Fourier Transform Infrared Spectroscopy (FTIR), Scanning Electron Microscopy (SEM), water uptake and drug release. The hydrogel exhibits high shear modulus, high gel strength. The hydrophilic silicone-based soft biomaterial enables controlled drug delivery with tunable diffusion behavior and stable encapsulation of drugs.

To overcome these limitations, the present invention provides an injectable, 3D extrusion-printable and biocompatible hydrogel. The synergistic blend of amine-functionalized silicone elastomers and hydrophilic monomers, structured into a semi-interpenetrating polymer network (semi-IPN). This hybrid formulation is further enhanced by the incorporation of cellulose nanocrystals (CNCs), which impart desirable shear-thinning and yield stress properties, allowing for precise and stable 3D printing using a UV-assisted microextrusion process.

The hydrogel composition leverages a synergistic combination of hydrogel and silicone materials, enhancing wearer comfort, sustained drug release, and mechanical strength. Notably, the hybrid hydrogels exhibit zero permanent set under cyclic compression, demonstrating exceptional elasticity and shape retention-properties not seen in conventional contact lens-compatible materials. The drug release follows a non-Fickian anomalous transport mechanism enabled by a semi-IPN, preventing the burst release commonly found in traditional systems. SEM reveals a microporous structure with tunable mesh size and crosslink density, allowing precise control over diffusion rates and oxygen permeability-both critical for ocular compatibility. The hydrogels also exhibit excellent long-term stability in aqueous media (PBS), and UV-visible spectroscopy confirms that the encapsulated drug remains chemically intact, preserving therapeutic efficacy. Furthermore, the fabrication process is streamlined through a single-step UV-curing and 3D printing method, enabling on-demand customization of drug-loaded geometries and offering superior efficiency and reproducibility compared to the multi-step processes reported in existing literature.

These features enable spatially controlled, long-term drug delivery directly on ophthalmic substrates such as contact lenses, with retained structural fidelity and sustained therapeutic performance. Additionally, the simplified, one-step curing and printing process allows for scalable, customizable manufacturing suitable for clinical deployment.

### BRIEF DESCRIPTION OF THE DRAWINGS

Embodiments of the invention are described in more details hereinafter with reference to the drawings, in which:

FIG. 1 shows a graphical illustration of the fabrication process for preparing hydrophilic silicone-based hydrogel and the plausible mechanism of gelation;

FIG. 2A Shear viscosity evaluation of the prepared inks and their shear thinning character (FIG. 2B) evaluation of printability using several sets of printing parameters; under-, proper-, and over-gelation (FIG. 2C) printed diagram of an eye-model (FIG. 2D-2G) different types of printed structures showing printability of the ink (FIG. 2H) printed over contact lens throughout the periphery of the lens magnified image of the printed zone in lens;

FIG. 3A FTIR spectrum of aminosilicone (AS)-based hydrogel (FIG. 3B) Experimental equilibrium swelling (ESRexpt), gel % and yield % of the prepared hybrid hydrogels;

FIG. 4A Frequency sweep experiment of the hybrid hydrogels (FIG. 4B) the gel strength of the fabricated hybrid gels (FIG. 4C) shear viscosity vs shear rate plot of the hybrid hydrogels with monomer ratio variation (FIG. 4D) shear viscosity vs shear rate plot of the hybrid hydrogels with AS variation (FIG. 4E) magnified plot for calculating the onset of rupture of the hybrid hydrogels (FIG. 4F) histogram plot of the onset of rupture point (FIG. 4G) damping behaviour of the hybrid hydrogels with monomer ratio variation (FIG. 4H) damping behaviour of the hybrid hydrogels with AS variation;

FIG. 5A SEM image of poly (AM-HEMA) copolymer hydrogel FIG. 5B magnified image of poly (AM-HEMA) copolymer hydrogel (FIG. 5C) SEM image of poly (AM-HEMA)/AS hybrid hydrogel (G3) (FIG. 5D) magnified image of poly (AM-HEMA)/AS hybrid hydrogel (G3) hydrogel (FIG. 5E) SEM image of poly (AM-HEMA)/AS hybrid hydrogel (G5) (FIG. 5F) magnified image of poly (AM-HEMA)/AS hybrid hydrogel (G5) hydrogel;

FIG. 6A Time dependent water uptake behaviour of the hydrogels and pseudo 2nd order kinetic model fittings of the hydrogels (FIG. 6B) Stability assessment of the hydrogel in PBS environment for 10 days (FIG. 6C) Diffusion parameters assessment of the different hydrogels (FIG. 6D) Diffusion exponents of the various hydrogels (FIG. 6E) Permeability of the hydrogels obtained from water uptake behaviours;

FIG. 7A Typical stress vs strain plot in compression mode (FIG. 7B) Histogram plot of the ultimate compressive strength (UCS) and Young's moduli of the hybrid hydrogels with increasing silicone content (FIG. 7C) Cyclic loading-unloading plot of the G5 hybrid hydrogel;

FIG. 8A Cumulative release percentage of the drug molecules from the hybrid hydrogels (FIG. 8B) Time dependent plot of Korsmeyer-Peppas model for drug release plot (FIG. 8C) Different release exponent and their mode of release mechanism (FIG. 8D) spectra of pure drug and after release (in water) in different times;

FIG. 9 depicts cumulative drug release profiles of the hybrid hydrogels in terms of drug eluded from the composted in ambient condition; and

FIG. 10 depicts Korsmeyer-Peppas derived constants (K_{KP}) of the hybrid hydrogels.

### DETAILED DESCRIPTION

While silicone elastomers have found widespread usage in the biomedical industry, 3D printing them has proven difficult due to the material's slow drying time, low viscosity, and hydrophobic properties.

In addition, traditional hydrogels offer biocompatibility and drug encapsulation capabilities but suffer from low oxygen transmissibility and poor mechanical resilience. Conversely, silicone-based elastomers provide excellent oxygen permeability but are typically hydrophobic, mechanically unstable in printed form, and incompatible with uniform drug release profiles.

Therefore, the present invention provides a novel injectable and 3D extrusion-printable hydrophilic silicone-based hydrogel, designed specifically for ophthalmic drug delivery. This hybrid material overcomes key limitations in current 3D-printed hydrogel systems by combining the mechanical flexibility and oxygen permeability of silicone elastomers with the biocompatibility and drug-retaining capacity of hydrophilic polymers, resulting in a semi-IPN with tunable diffusion characteristics and exceptional structural integrity.

In contrast to traditional hydrogel or silicone-based matrices, the present invention integrates amine-functionalized silicone with poly(acrylamide-co-2-hydroxyethyl methacrylate) (poly(AM-co-HEMA)) via UV-assisted microextrusion printing, forming a robust hydrogel system. The thixotropic properties of the ink, derived from CNCs, ensure both extrudability and structural fidelity during layer-by-layer deposition, while UV-initiated free radical polymerization provides rapid and effective in situ gelation.

The specific combination of each constituent material into a structurally resilient, shear-thinning, and printable hydrogel composition represents a significant advancement over the prior art. This hybrid material successfully merges the drug-retaining and biocompatible characteristics of hydrophilic polymers with the mechanical flexibility and oxygen permeability of silicone elastomers-features not previously achieved in a single hydrogel system compatible with extrusion-based 3D printing.

Unlike prior systems that suffer from either rapid collapse during extrusion or poor post-printing mechanical strength, this hybrid ink formulation exhibits shear-thinning behavior, rapid solidification, and high gel strength, confirmed through rheological analyses including frequency sweep, damping behavior (tan δ), and onset of rupture testing. These mechanical properties were found to be tunable based on the AM/HEMA ratio and AS concentration, allowing the tailoring of viscoelastic profiles to meet application-specific demands.

The silicone phase imparts high oxygen transmissibility and elastic recovery, while the polar copolymer network of poly(AM-co-HEMA) enables effective drug encapsulation and sustained release. CNCs act as rheological modifiers, imparting thixotropic behavior and enhancing shape fidelity during the printing process. The resulting hydrogel displays a tunable viscoelastic profile, with key parameters such as mesh size, elastic modulus, and drug diffusion rate directly controlled by varying the AM/HEMA ratio and AS concentration. Notably, rheological analysis demonstrates high shear modulus, zero compression set under cyclic loading, and non-Fickian anomalous diffusion-all indicative of superior mechanical and functional performance not predicted by the prior art.

The hydrogel is fabricated using a streamlined, one-step UV-assisted microextrusion printing process. This method represents a significant improvement in manufacturability compared to conventional multi-step protocols used in hydrogel or silicone-based drug delivery systems. The ink formulation exhibits shear-thinning behavior, making it highly suitable for layer-by-layer deposition during 3D printing, while the incorporation of CNCs ensures extrusion stability and structural fidelity. Upon deposition, rapid in situ gelation is triggered by UV-initiated free radical polymerization, eliminating the need for post-printing crosslinking or thermal curing.

Unlike traditional hydrogels such as gelatin-methacrylate (GelMA) or alginate, which often suffer from low mechanical strength, poor shape retention, and unpredictable drug diffusion, the present invention maintains high mechanical integrity and allows precision shaping of complex geometries. The entire process—from material extrusion to final curing—can be completed in a single, continuous step, offering enhanced scalability, reproducibility, and cost-efficiency for biomedical manufacturing.

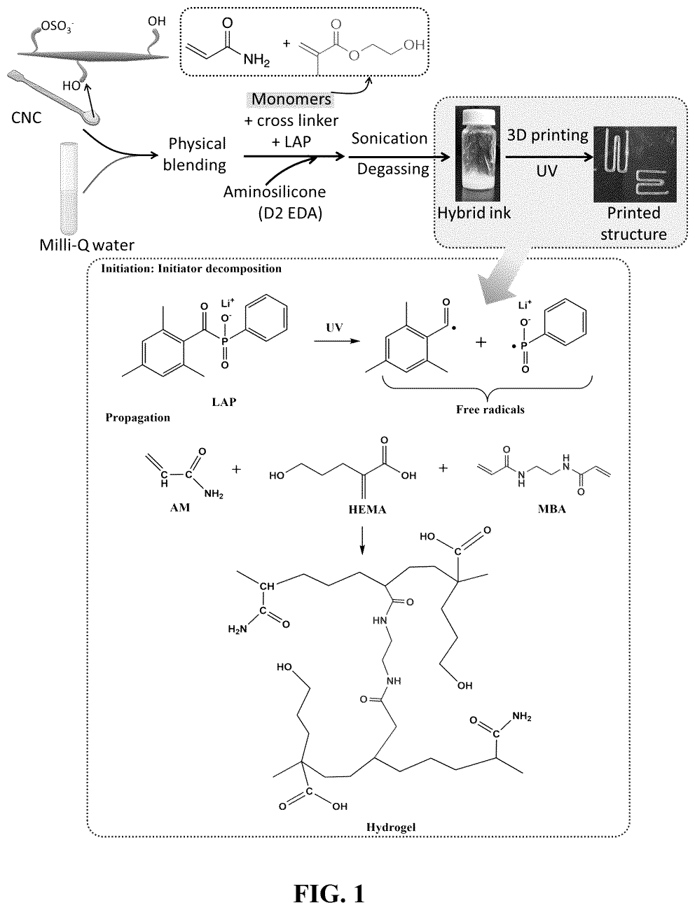

An overview of the fabrication process for the hydrophilic silicone-based hydrogel is presented in FIG. 1. CNCs are used as thioxotropic agent forming physical gel in presence of water resulting shear thinning behaviour of the hybrid ink. As CNCs are produced sulphuric acid hydrolysis, it consists abundant negative charges throughout the surface. The CNCs regulate the rheological characteristics of the ink, making it possible to generate a yield stress adequate for the temporary fixation of an extrusion-printed layer.

In order to better understand how ink behaves during the various stages of printing, rheology measurements are carried out. To prevent ink dripping from the syringe during non-printing stages, ink viscosity must be sufficiently high at a low shear rate. But at the time of syringe piston movement, viscosity must decrease promptly to allow extrusion. The gelation occurs here by UV triggered free radical polymerisation where lithium phenyl-2,4,6,-trimethylbenzoyphosphinate (LAP) is the photo-dissociable initiator. At the initiation stage LAP produces free radicals followed by 1 vinylic polymerization of AM, HEMA, and N,N′-Methylenebisacrylamide (MBA). AS is the non-crosslinked phase here entrapped into the gel matrix forming semi-interpenetrating polymeric network system.

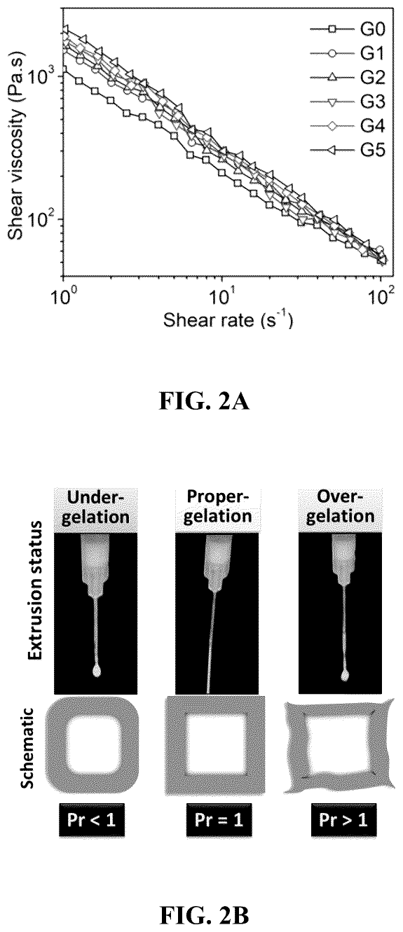

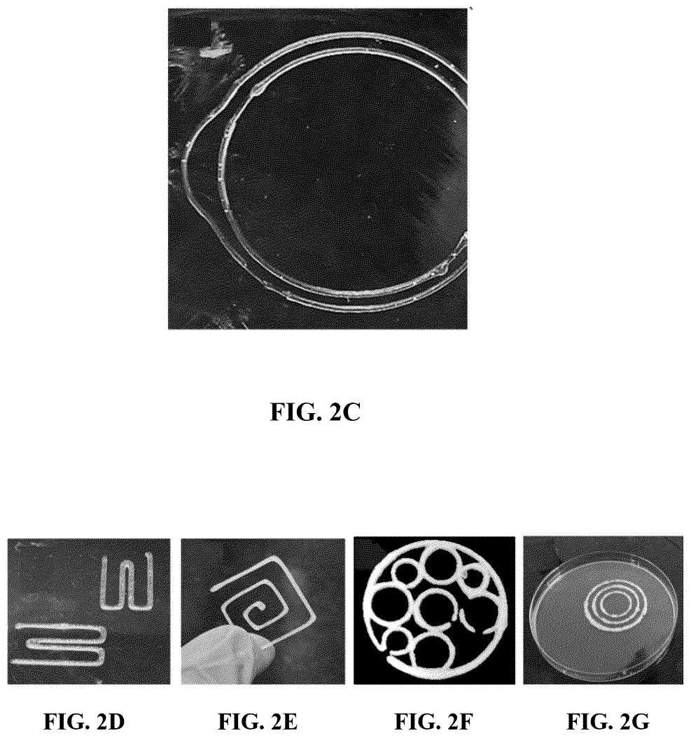

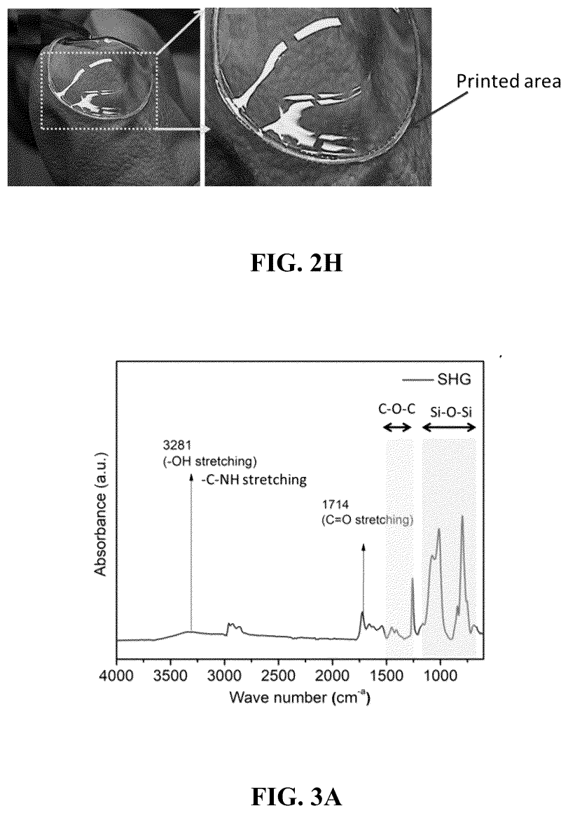

Referring to FIG. 2A, shear thinning occurred in all the formulations with very subtle changes. For perfect gelation or printability, the channels of the constructs would be connected in a square shape, and the Pr value would be 1. The level of gelation of the ink was found to be higher the higher the Pr value. The degree to which the ink gelled was lower the lower the Pr value. ImageJ (National Institute of Health) software was used to measure the perimeter and area of interconnected channels in printed structures to figure out the Pr value of each combination of printing parameters. Many methods were used to determine whether or not an ink could be printed, including rheology, ink state evaluations at the needle tip, and the stability of the printed multilayer structure.

FIG. 2B depicts the three possible gelation states for printed ink: under-gelation, proper-gelation, and over-gelation. Under-gelation printing results in droplet morphology at the nozzle tip and ink fusing at the cross site, making it impossible to produce a mechanically sound 3D construct. Nevertheless, when the ink was over-gelled, its s hape became readily fragmented, displaying uneven filaments and linked channels. The printability of ink G4 was found to be around 0.92, which is very close to value 1, and as would be expected, for direct ink writing technology.

FIGS. 2C-2G depict the various printed structures made from the hybrid ink. All the structures showed intricate shapes without any flow of the extruded filaments which also support the printability of the hybrid inks.

A primary application of these hydrogels lies in ophthalmic drug delivery, specifically in the development of therapeutic contact lenses. The hybrid material can be printed directly onto or into contact lens substrates, particularly along the lens periphery, enabling patient-specific designs without compromising lens geometry or comfort. The hydrogel's strong adhesion to the lens surface, combined with its zero permanent set and sustained drug release properties, makes it uniquely suited for long-term ocular applications. For instance, contact lens integration is made feasible via precise printing of hydrogel structures along the lens periphery, achieving strong adhesion without distorting the CL geometry. This feature significantly broadens clinical utility, enabling patient-specific therapeutic contact lenses that sustain drug release over prolonged periods.

The semi-IPN structure enables tunable, non-Fickian diffusion kinetics, providing controlled and sustained delivery of therapeutic agents over extended durations. UV-visible spectroscopy confirms chemical stability of encapsulated drugs, ensuring consistent efficacy throughout the treatment period. These features significantly outperform prior systems that rely on burst release, bulk-loaded drug reservoirs, or incompatible silicone matrices.

For printing over CLs, initially a circular CAD model was drawn with matching the outer diameter of the contact lens (14.3 mm). Then the CL was taken from the lens solution followed by gentle soaking into delicate task wipers. After complete removal of surface water, the CL was placed onto a glass support followed by printing as mentioned earlier. After printing the printed-CL assembly was kept under UV-light (5V, 2A, 365 nm) for around 6 s and dismantled from the glass support. The printed CLs have been displayed in FIG. 2H.

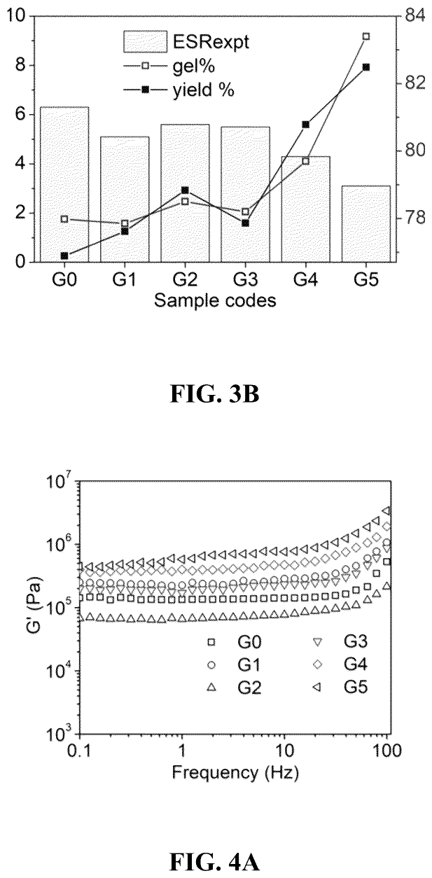

FIG. 3A shows the FTIR spectrum of the synthesized hydrogel. The spectrum shows major absorption peak at the range of 3350-3281 cm⁻¹ which is due to the stretching vibration of hydroxyl groups present in HEMA and —NH₂ groups of silicone. Both the monomers AA and HEMA have chaparetric stretching absorption of —C═O at around 1714 cm⁻¹. Another significant peak at around 2900 cm⁻¹ is also observed which is due to stretching vibration of aliphatic C—H bonds present in polymer chains. The confirmation of silicone phase is supported by their characteristic peaks in the range of 800-1100 cm⁻¹ which are due to the presence of Si—O—Si linkages.

FIG. 3B is the histogram plot of the different physical parameters related to the synthesis. These synthesis variables i.e. equilibrium swelling ratio (ESR), gel %, and yield % are dependent on the monomer ratio and hydrophilic silicone content. The ESR is dependent over the polarity of the hydrogel. It is seen from the plot that the ESR values were decreasing when monomer ratios were changed. For G1, the ESR value was lowered significantly due to combinational effect of AS and lowering of AA content. But for G2, the ESR was increased due to presence of more amount of AA. For G3 to G5, the ESR values show a decreasing trend due to increase in AS content. As AS does not posses sufficient hydrophilicity, thus the water imbibition through the hydrogel network becomes limited. For the gel % and yield % data, monomer ratio affects slightly but AS concentration affects drastically. Normally gel % is getting higher when least amount of leachable fractions are present in the gel metrics in forms of oligomers and unreacted monomers. But with AS content, the arrest of the silicone macrochians affect very negligible leaching resulting improvement in gel %.

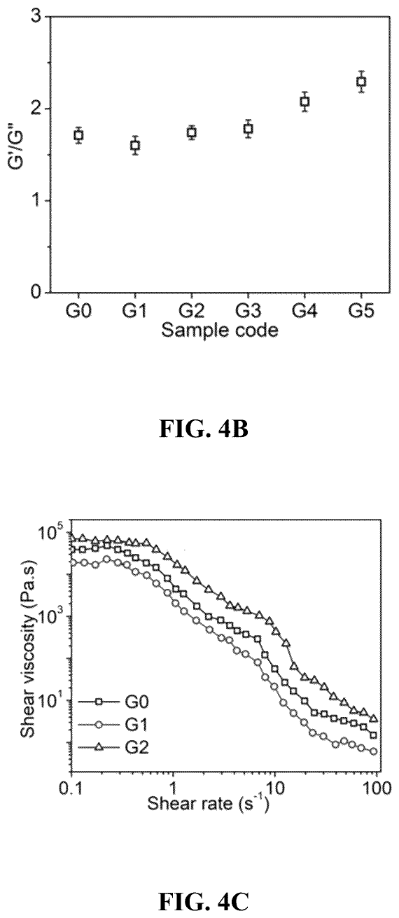

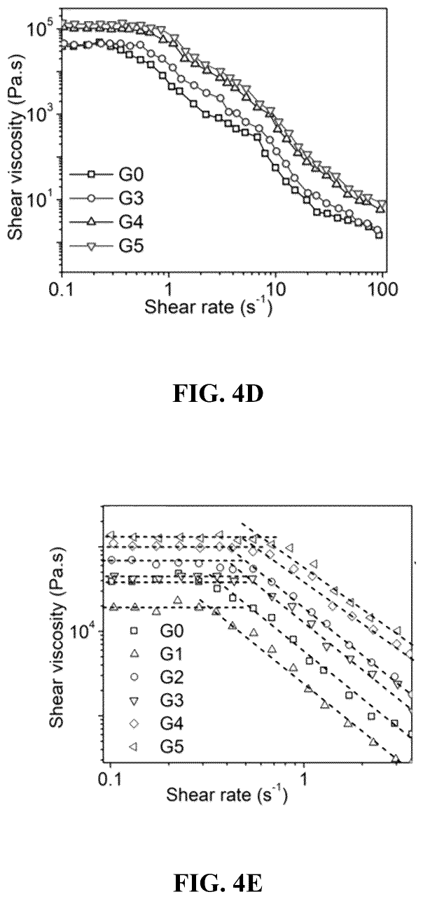

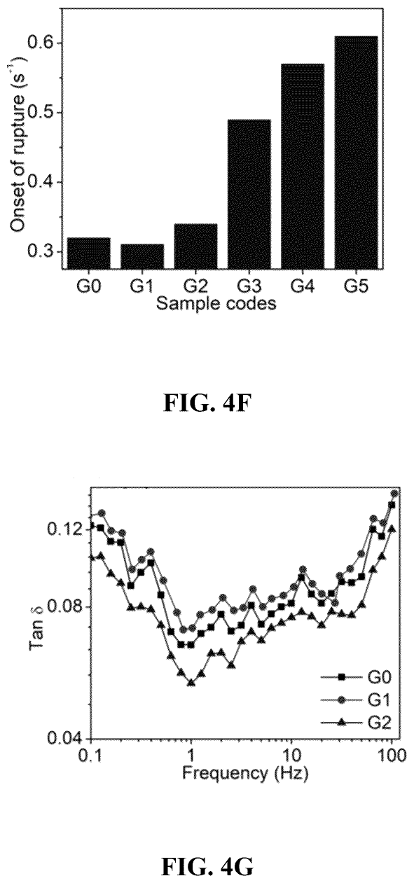

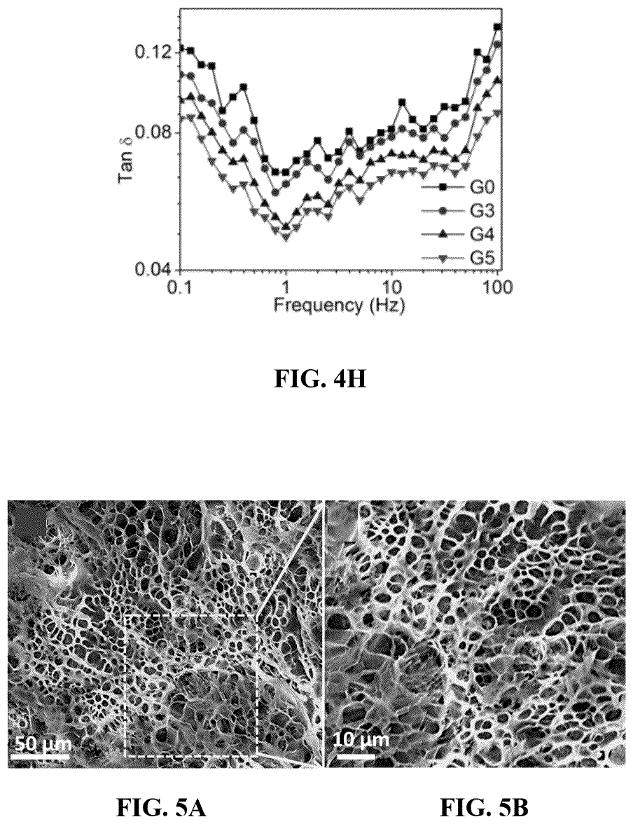

The hydrogels in swelled state are tested against shear force to evaluate their gel strength. FIG. 4A shows the frequency sweep experiment where the elastic moduli of the fabricated hydrogels were plotted. As the major strength bearing component is CNCs here, the main cause of the high elastic component (G′) for all the gels is justified. But the fluctuations in the G′ are due to the cause of other compositional factors because CNCs mass fraction is kept constant here. The G′ value was initially decreased from G0 to G1 due to the changing of monomer ratio. In case of G1, the AM content was comparatively low than G0 which might be a cause of lowering of extent of H-bonding and polar-polar interactions. But when the AM content was increased for G2, the G′ value increased. After addition of AS, the formation of semi interpenetrating polymer network has been formed. Semi-IPNs architecture generally provides better resistance to flow against shear force. As a result, the gel strength was increased with increasing the AS content.

Moreover, the chance of polar-polar interactions, H-bonding among the amino groups and copolymer pendent groups, and hydrophobic association between silicone macrochains could also impose better stiffness of the gel matrices. The gel strength was also calculated from the ratio of the elastic to loss/viscous modulus (G′/G″) as shown in FIG. 4B. It is evident from the plot that the gel strength for G1 was low compared to G0 but for G2, the gel strength was increased due to the high AM/HEMA ratio. Furthermore, the introduction of AS phase, the semi-IPN morphology is more prominent resulting better gel strength. The point of network rupture is another way to accumulate information of how the hydrogels behave under variable shear rate. FIGS. 4C-4D are the plots showing the shear rate dependency of the fabricated hydrogels. The AM/HEMA ratio also played here similar kind of trend as of the frequency sweep experiment. The onset of rupture was calculated from the liner extrapolated lines of the linear viscoelastic region (LVR) and falling region (FIG. 4E). The right shift of rupture point clearly designates the delayed network rupturing for higher AS content. The values of the onset of rupture are plotted in the histogram in FIG. 4F for clear understanding.

In FIGS. 4G-4H, tan δ vs frequency has been plotted to evaluate the damping behaviour of the hybrid hydrogels. Tan δ is a measurement of how much energy a material can absorb before it starts to release that energy in other forms. It quantifies the lag in time between an impact stress and the subsequent force applied to the supporting body. The highest tan δ values are seen in hydrogels where the largest phase is reaching to 90°. When external stress is incident to the surface of the material, there is stress dissipation perpendicular to the impact surface. CNCs, being anisotropic nanofiller, also perform fast stress transfer efficiently. The tan δ is sometimes referred to as the loss Factor due to the conversion and dispersion of the impact force's energy into a safer form. Hence, the tan delta is a measure of a material's practical damping properties. The larger the tan δ, the stronger the damping coefficient which means better the material's ability to absorb and disperse energy efficiently. Here, the damping behaviour also shows compositional dependency. When AM/HEMA ratio was increased the damping behaviour showed low value due to lowering of lossy part of the copolymer. The lossy part is normally affected because of extensive H-bonding. For high AM content, the pendent functionalities are more prone to H-bonded inside hydrogel matrices resulting lowering of lossy features of the hydrogels. Similarly, when the AS content was increased the elastic part is dominated over the lossy part and again the damping behaviour was minimized. It is also a proof of elastic network and interpenetrating polymer network formation.

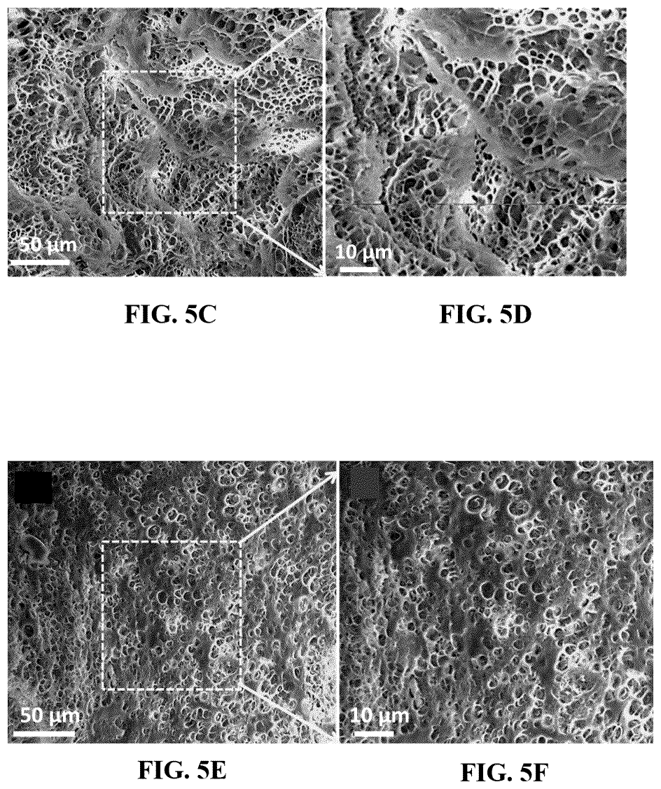

The porous morphology of the prepared hydrogels are shown in FIGS. 5A-5F. Freeze dried hydrogel specimens show highly porous structure which is called dried hydrogel or xerogel resembling highly fluffy and spongy. For comparison of their morphology, three types of hydrogels are taken; copolymer hydrogel (G0, without AS), low AS content hybrid hydrogel (G3), and highest AS content hydrogel (G5). All the hydrogels have porous kind of morphology which is the advantage of molecular diffusion through the matrix. For copolymer hydrogel (G0) and low AS containing hydrogel (G3), the cell walls of the pores are comparable in thickness. But after increasing the AS content (here G5), the cell wall thickness was increased which supports better gel strength. Moreover, the lowering of porosity is also visible which affects the molecular imbibition through the matrices.

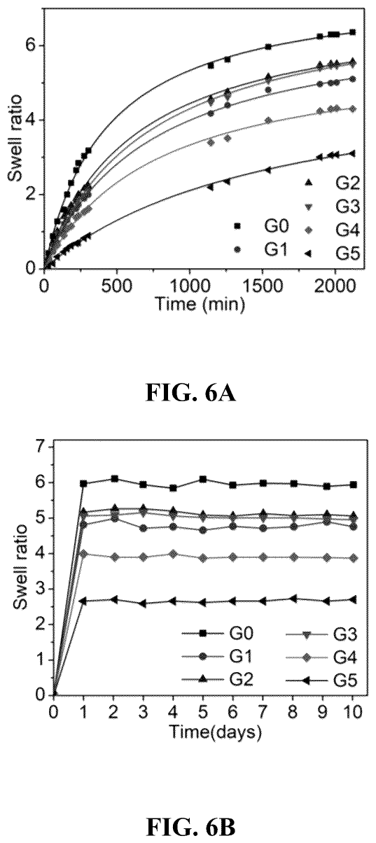

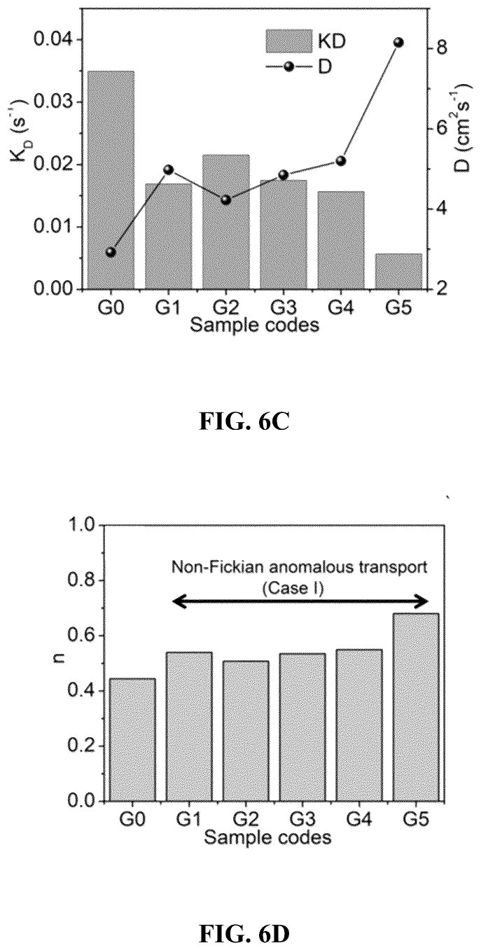

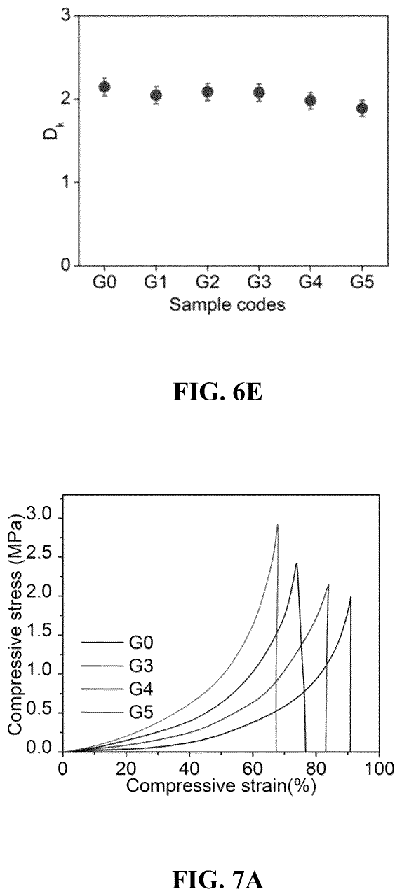

FIG. 6A displays a time dependent scatter plot of the water uptake data, which was immediately fitted to the following pseudo second order rate Equation (1):

$${S}_{t} = \frac{{S}_{ecal}^{2} {k}_{S 2} t}{1 + {k}_{S 2} {S}_{ecal}} = \frac{{r}_{0} t}{1 + {k}_{S 2} {S}_{ecal}} , \qquad ( 1 )$$

where r₀ is the initial swelling rate, S_{ecal} is the estimated ESR, and kₛ₂ is the corresponding rate constant for the second order.

The Levenberg-Marquardt (L-M) algorithm (Origin-8 software) adjusted the rate constant (k_{S2}) and starting rate of swelling (r₀) by iteration utilising the chi square (χ²) and F values. Table 2 displays the experimental (S_{wexpt}), computed (S_{wcal}), rate of swelling (r₀), and 2nd order rate constant (k_{S2}) of swelling, as well as other statistical parameters.

TABLE 2
Swelling, diffusion, and network parameters of the hydrogels

| | ESRₑₓₚₜ/ | | | | | |
|---|---|---|---|---|---|---|
| Hydrogel | ESR_{cal} | | | | | |
| codes | (g/g) | K_{S2} × 10₃ | r²/χ² | K_{D}/n/D × 10⁶ | χ²/r² | M_{c}/ρ_{c} × 10⁻²²/ç |
| G0 | 6.3/7.6 | 0.3023 | 0.998/0.00694 | 0.034/0.44/2.91 | 0.00288/0.975 | 9.34/7.03/0.822 |
| G1 | 5.1/6.8 | 0.2043 | 0.999/0.00367 | 0.016/0.53/4.97 | 0.00213/0.984 | 7.90/8.23/0.753 |
| G2 | 5.6/7.1 | 0.2252 | 0.998/0.00498 | 0.021/0.51/4.22 | 0.00122/0.99 | 9.18/7.48/0.813 |
| G3 | 5.5/7.3 | 0.1919 | 0.999/0.00408 | 0.017/0.53/4.84 | 0.00255/0.991 | 9.37/7.46/0.822 |
| G4 | 4.3/5.8 | 0.2203 | 0.998/0.00475 | 0.016/0.55/5.19 | 0.000934/0.992 | 9.30/8.63/0.771 |
| G5 | 3.1/5.2 | 0.1177 | 0.997/0.00181 | 0.006/0.68/8.15 | 0.000475/0.0996 | 8.53/8.54/0.781 |

From Table 2, it can be seen that the values of S_{wexpt} and S_{wcal} are, in fact, quite near to one another. Furthermore, the values of r² are shown to be trending toward unity, while low values of χ² are established by the data fits.

Investigating how drug molecules diffuse into hydrogel and are absorbed is crucial. The diffusion process and diffusion parameters of the hydrogels, including the diffusion exponent (n), diffusion coefficient (D), and diffusion constant (k_{D}), were evaluated by fitting the swelling data to Equations (2) and (3):

$$F = \frac{{w}_{t}}{{w}_{e}} = {k}_{D} {t}^{n} \qquad ( 2 )$$

$$D = π {{r}^{2} ( \frac{{k}_{D}}{4} )}^{\frac{1}{n}} , \qquad ( 3 )$$

Hydrogel samples are cylindrical; hence the radius r is used to express the water absorption percentage, F. To study the diffusion properties of composite gels, a nonlinear regression and data fitting were carried out. Diffusion properties (k_{D}, n, and D) of the hydrogels are also reported in Table 2.

The stability of hydrogels in water is another essential characteristic for assuming the dimensional and volume fluctuation behaviour. FIG. 6B depicts that the hydrogels did show any drastic change in their swelling behaviour as their swelling ratios are quite kept constant throughout the experiential tenure of 10 days. The k_{D} and D value show inverse trend for the hydrogels (FIG. 6C). The D values of the hydrogels have direct correlation with their AS content and monomer ratio. The D value is affected by the internal morphology especially the porosity of the gel network. For high AM/HEMA ratio, there is a chance of extensive H-bonding which could restrict the molecular movement (here water) throughout the hydrogel.

Similarly, for AS incorporated hydrogels, there is again an increment in interchain spacing among the AM-HEMA copolymer chains resulting formation of larger pores. This larger pore facilitates the mass diffusion. The diffusion nature is also evaluated by measuring the diffusion exponent from non-linear curve fitting as shown in FIG. 6D. The diffusion exponents are ranging from 0.5 to 0.7 which implies the non-Fickian anomalous transport mechanism. This infers the rate of macrochain relaxation rate of the hydrogels and the rate of water imbibition (diffusion) are comparable. But for AM and HEMA based copolymer hydrogel, the n value was around 0.5 which signifies Fickian case-I diffusion. In this case, rate of hydrogel chain relaxation is dominant over the rate of diffusion. The oxygen permeability (D_{K}) is also evaluated from the water uptake data as shown in FIG. 6E. The empirical equation regarding this is:

$${D}_{K} = 1.67 {e}^{0.0397 ESR} . \qquad ( 4 )$$

The assumption for this working equation is only applicable when the imbibed molecule is water. That's why the hydrogel's D_{K} calculation was performed against deionized water only. The oxygen transport values are almost comparable for all the hybrid hydrogels which could infer its applicability as an ocular delivery matrix.

The average molecular weight between crosslinks, M_{c}, and mesh size, as determined by neutron scattering or quasi-elastic light scattering, are commonly used to describe the gel network. Based on Flory and Rehner's network theory, the following equation yields M_{c}:

$${M}_{c} = - \frac{{V}_{s} {ρ}_{p} [ {φ}_{p}^{1 / 3} - \frac{{φ}_{p}}{2} ]}{ln ( 1 - {φ}_{p} ) + {φ}_{p} + {χφ}_{p}^{2}} , \qquad ( 10 )$$

From its density (0.98 g cm⁻³) and molecular weight (18 g mol⁻¹), the molar volume of water, Vₛ, was determined at the experimental temperature (25° C.) (18.18 cm³ mol⁻¹). The density, ρₚ, of the hydrogel was determined using its mass and volume. For equilibrium swelling of m_{w} g water/g dry hydrogel sample, polymer volume fraction in swollen gel under equilibrium, φₚ will be:

$${φ}_{p} = {( 1 + \frac{{ρ}_{p}}{{ρ}_{i}} {m}_{w} )}^{- 1} , \qquad ( 11 )$$

where ρₚ and ρᵢ stand for polymer density and solvent (water), respectively. Equation (12) is used to calculate the interaction parameter between polymeric hydrogel and water.

$$χ = \frac{{V}_{p}}{3} + 0.5 . \qquad ( 12 )$$

The crosslink density (ρ_{c}) of a hydrogel is obtained as

$${ρ}_{c} = \frac{{ρ}_{p} {N}_{A}}{{M}_{c}} , \qquad ( 13 )$$

where N_{A} is Avogadro's number (6.023×10²³/mol). The mesh size (ç in Å) of the swollen gel is calculated from the following Eq:

$$ς = {[ {C}_{n} ( \frac{2 {M}_{c}}{{M}_{r}} ) ]}^{\frac{1}{2}} {Ιϕ}_{p}^{\frac{1}{3}} . \qquad ( 14 )$$

The Flory's characteristic ratio, Cₙ, was obtained from literary works and C—C bond length; ‘T’ was assumed as 1.54 Å.

The network parameters showed especially the crosslinked density of the hybrid hydrogels shows composition dependency. The network becomes tighter after incorporation of AS content. Moreover, the monomer ratio also played a significant role here. When AM content is higher the gel becomes tighter and denser as shown from the Table 2 data. The mesh size was also calculated from eq. 14 which shows lowering of mesh sizes with increasing the AS content. This could be due to the formation of extensive entangled polymer network in the hydrogel.

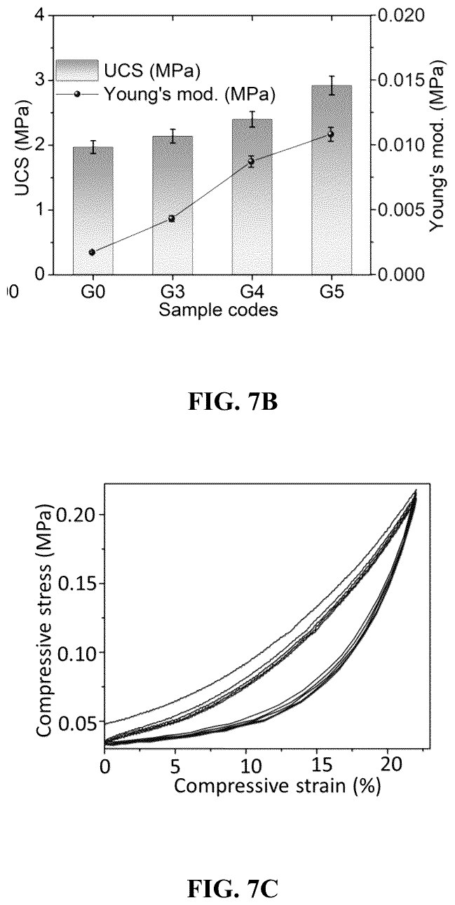

Furthermore, the mechanical properties of the hybrid hydrogels were conducted by uniaxial compression testing as shown in FIG. 7A. The typical stress-strain plot shows that the ultimate compressive stress (UCS) increases with increasing the silicone elastomer content. The improvement of compressive strength might be due to formation physical networking among the silicone and copolymer chains. The amino functionalities of silicone elastomer are prone to interact with hydrogel pendent functionalities (—COOH, —OH). This offers compact network formation and high degree of crosslinking.

Additionally, the chemical structure of silicone elastomers allows them to undergo a high degree of deformation before failure. This is due to the highly flexible nature of the silicone-silicone bonds in the material, which can stretch and bend without breaking. Besides this, the load bearing capacity and fast stress dissipation of CNCs are also playing major role. FIG. 7B depicts the ultimate compressive strength and Young's moduli of the hydrogels which shows gradual improvement with increasing the AS content.

Besides these, to ensure the load withstanding capability of the hybrid hydrogels, cycling loading-unloading performance were done of the highest AS containing hydrogel G5) as shown in FIG. 7C. The cyclic loading-unloading test was done within 22% deformation and it is shown that there were no compressions set behaviour. Compression set is a measure of a material's ability to recover its original shape after being subjected to a compressive force for an extended period of time. A low compression set means that the material is able to recover its original shape more effectively after being compressed. The combination of the silicone backbone and the hydrophilic component in silicone-based hybrid hydrogels results in a material with a unique set of mechanical properties that make it highly resistant to compression set. The extent of polar-polar interaction normally improves their elastic character whereas the elastomeric nature of silicone imparts viscoelastic counter parts of the matrix. Zero compression set character of the hybrid hydrogel could promote this as a soft biomaterial with rubber-like consistency.

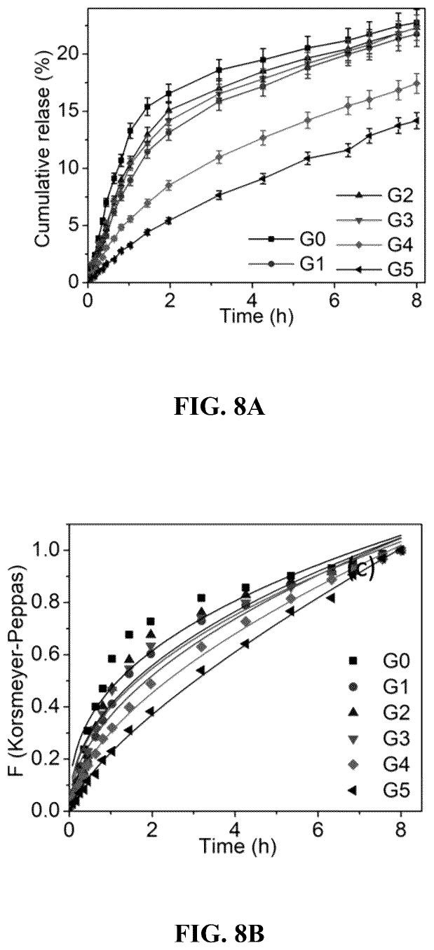

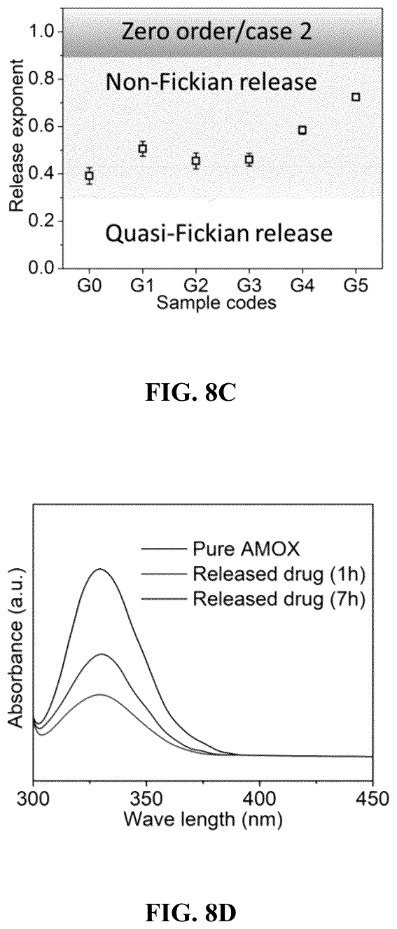

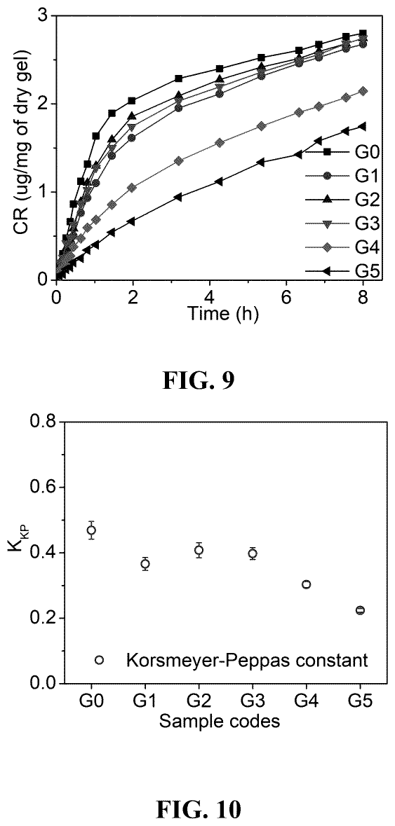

The cumulative drug release from the hydrogels is plotted against time in FIG. 9. The plot from FIG. 8A shows the release percentage data of the model drug (here amoxicillin hydrochloride). It is observed from the cumulative release data that G0 hydrogel shows initial burst release and about 15% of the release was done within first 1 h. Burst release is happened because of the high concentration gradient between hydrogel matrices and release environment. This release was little restricted when AS was incorporated. It is already established from the diffusion behaviour of the hybrid hydrogels that with increasing the AM content, the extent of polar-polar interaction is increased resulting formation of physical crosslinking. Thus, diffusion of drug molecules was also throttled because of this. As a result, the lowering of cumulative release is observed. The control of the drug release profiles is also achieved after incorporation of hydrophilic elastomeric phase (AS). The release is more controlled with increasing the AS content which could be inferred as the formation of tight network gel. As drug release proceeds, the drug's concentration in the release environment goes up and the concentration gradient of the drug between the hydrogel matrix and the release medium goes down. The slowing of drug release at a low concentration gradient is caused by the remaining drug being trapped in the gel network.

To evaluate the release kinetics and mechanism, the release data has been fitted into Korsmeyer-Peppas model as shown in eq. 15:

$${F}_{D} = \frac{{m}_{D t}}{{m}_{D e}} = {K}_{K P} {t}^{n} , \qquad ( 15 )$$

where m_{Dt} is the amount of drug released at time t and m_{De} is the amount released at infinity (at equilibrium); K_{KP} is constant of the model based on the structure and geometry of the dosage form. The diffusion exponent, ‘n’ shows how the drug is released.

The fitting of the cumulative release data is shown in FIG. 8B. The kinetics of drug release is represented by the values of ‘n’ in the Korsmeyer-Peppas (K_{KP}) model. When the diffusion rate is substantially slower than the relaxation of the polymer chains in the gel, drug release is dictated by case-I Fickian diffusion for ‘n’ values up to 0.5. When the rate of diffusion is significantly larger than the rate of chain relaxation (n=0.5), we get case-II or relaxation-controlled diffusion. When both the rate of diffusion and the rate of relaxation are equivalent, the drug release is dictated by anomalous case-III non-Fickian diffusion for n values higher than 0.5 and up to 1 (0.5 n 1). This occurs when n is greater than or equal to 1. The K_{KP} and the ‘n’ values both are represented in FIGS. 10 and 8C, respectively. For most of the hybrid hydrogel systems, the ‘n’ values are in the range of 0.5-0.72. This signifies the restricted relaxation of polymeric chains inside gel matrix in present of AS and CNCs due to lowering of degrees of freedom. After a drug-loaded gel sample is submerged in water, the drug molecules are simultaneously desorption from the gel and released into the water. In the case of uncrosslinked polymers, the glass transition temperature decreases as water permeates the gel network, and the drug molecules diffuse out through the swollen rubbery areas to the release medium through a swelling-controlled non-Fickian diffusion process. Crosslinking, interpenetration between the AS phase and the poly (AM-HEMA) copolymer phase, and the presence of CNCs filler all contribute to the lack of substantial molecular relaxation in the fabricated hydrogel, which in turn results in Fickian diffusion.

After being encapsulated in the gel, the drug molecule can go through an undesirable chemical transformation due to its interaction with the gel matrices. This outcome is not acceptable. The drug's chemical composition should not change after it has been released into water, ensuring that it retains the same therapeutic efficacy as before. The UV absorption of a chemical can be used to judge how pure it is. So, after studying how the drug was released, the drug-containing water was put through UV absorption. FIG. 8D shows the UV spectra of the original drug and the drug that was released into water. At a wavelength of 330 nm, the spectra of both the pure drug and the released drug are almost similar, showing that there was little to no change in the chemistry and bioactivity of the medication between loading and release.

In this invention, a series of hydrophilic silicone-based 3D printed hydrogels have been developed for drug delivery applications. The hybrid ink was formulated by combining AS and copolymer phase associated with CNCs as shear thinning agent. The UV-curing hydrogel showed AS dependent water uptake and diffusion behaviour. The thin peripheral printing of the hydrogel was successfully carried out over contact lens. The mechanical properties of the hydrogels were also performed in uniaxial compression model which showed improvement of compression strength with increasing the AS content.

Moreover, the hydrogel was also passed the cyclic compression test without any residual strain/deformation. The SEM micrographs provide information about their porous microstructure which could also support their diffusion driven drug release. Time dependent cumulative ocular drug release from the hydrogels was controlled by altering their compositional ingredients which resulted delayed release behaviour (around 14% release within first 8 h). The release mechanism was also predicted according to the physical models and showed it lies in the non-Fickian domain. The findings define a novel class of semi-interpenetrating polymeric hydrogels based on silicone, with desirable properties including quick curing, shear-thinning, zero compression set, and controlled drug delivery.

Beyond ophthalmology, the hydrogel system offers broad potential across various medical domains including dermatology, chronic disease management, and infectious disease treatment. Its combination of printability, biocompatibility, oxygen permeability, and customizable drug release establishes this invention as a transformative platform for targeted therapy delivery. This offers opportunities in various medical sectors, including ophthalmology, dermatology, chronic and infectious diseases.

### EXAMPLE

### Example 1-Materials and Methods

### Material

Acrylamide (AM), 2-Hydroxyethyl methacrylate (HEMA), N,N′-Methylenebisacrylamide (MBA), lithium phenyl-2,4,6,-trimethylbenzoyphosphinate (LAP) were purchased from Sigma Aldrich. Aminosilicone (Silamine D2 EDA) was procured from Siltech Corporation, Canada. Cellulose nanocrystal (CNC) was provided by Professor Michael K. C. Tam from the Department of Chemical Engineering at the University of Waterloo, Canada. All the aqueous solutions were prepared by using Milli-Q water (18.2 M (2 cm⁻¹).

### Ink Formulation

At first 10 wt % dispersion of CNC was prepared in double distilled water by sonication. The obtained CNC dispersion was mixed with other precursor materials. The monomer ratio and the AS concentration were varied as shown in the Table 1. All the mixtures were vortexed until homogenisation followed by defoaming under reduced pressure. The resulting ink mixtures were stored in a lightproof 10 mL disposable syringe.

TABLE 1
ink composition

| Ink | 10 wt % | AM | HEMA | AS | MBA | LAP |
|---|---|---|---|---|---|---|
| Codes | CNC (g) | (g) | (g) | (g) | (g) | (g) |
| G0 | 2 | 1 | 1 | 0 | 0.1 | 0.1 |
| G1 | 2 | 0.5 | 1.5 | 0.2 | 0.1 | 0.1 |
| G2 | 2 | 1.5 | 0.5 | 0.2 | 0.1 | 0.1 |
| G3 | 2 | 1 | 1 | 0.2 | 0.1 | 0.1 |
| G4 | 2 | 1 | 1 | 0.6 | 0.1 | 0.1 |
| G5 | 2 | 1 | 1 | 1.0 | 0.1 | 0.1 |

### 3D Printing of Hydrogel

Hydrogels were 3D-printed using a FlashForge Creator Pro (FlashForge, USA) with a 365 nm UV goose neck LED accessory. A 10 mL disposable syringe (EFD Nordson, USA) with a 25-gauge needle tip (0.221 mm) was used throughout the printing process. Concentric circular models have been designed in FreeCAD software and saved as STL files. Then the files have been converted into G-code files by ReplicatorG. The ink flow rate was adjusted by varying the extruding pressure from 16 to 22 psi to match with the nozzle's velocity of 30 mm/s. Continuous UV irradiation at 365 nm was kept during the whole printing process.

### Semi-Quantification of Printability

When the ink is at its optimal gelation state, the extruded filament displays a distinct morphology, one with a smooth surface and constant width in all three dimensions. This ensures that the manufactured structures have uniform grids and square holes. Here we use a square-based definition of printability (Pr) to describe how well the ink can be used to create physical objects.

$$P r = \frac{{L}^{2}}{16 A} , \qquad ( 16 )$$

where L and A stand for the perimeter and area, respectively.

### Example 2—Fabrication of Drug-Loaded Hydrogel

A formulation comprising 15-25 wt % of acrylamide (AM), 5-10 wt % of 2-hydroxyethyl methacrylate (HEMA), 10-50 wt % of aminosilicone (AS), 3-6 wt % of N,N′-methylenebisacrylamide (MBA) as crosslinker, and 3-6 wt % of lithium phenyl-2,4,6-trimethylbenzoylphosphinate (LAP) as photoinitiator is prepared. Cellulose nanocrystals (CNCs) are added at a concentration of 8-12 wt % to impart shear-thinning behavior. The final composition is stirred and degassed before transferring into a 10 ml syringe.

The hydrogel is printed using a microextrusion printer (FlashForge Creator Pro) equipped with a 25 gauge needle under UV illumination at 2.0-5.0 mW/cm².

The design is a concentric circular patch with a diameter of 4-20 mm and thickness of 0.3-0.6 mm, matching the curvature of a standard contact lens. Post-printing UV curing was conducted for 3-8 seconds.

The printed patch adhered well to the lens surface without distortion and demonstrated mechanical integrity under manual deformation. The gel maintained its shape during swelling and drug release tests.

### Example 3—Characterizations of the Hydrogel

### Yield and Gel Content of the Hydrogels

The water-soluble portion of the manufactured hydrogels was removed by rehydrating the prepared dried material (Wᵢ) to a consistent weight and then soaking it in water for a week while occasionally shaking the container. Then the water-insoluble hydrogels were dried (W_{d}) until its weight remained constant. The results for yield (%) and gel (%) are:

$$Gel \% = \frac{{w}_{d}}{{w}_{i}} \times 100 \qquad ( 17 )$$

$$Yield \% = \frac{{w}_{d}}{{w}_{p}} \times 100 \qquad ( 18 )$$

where Wₚ is the total weight of monomers and AS used in the gelation.

### Swelling Experiments

The hydrogel sample was submerged in deionized water at 24° C. until the swelling equilibrium was attained. Filter paper was used to remove the surface water that was only weakly attached to the hydrogel and then the weighed at various time intervals during the swelling experiment. Swelling ratio (SR) was estimated as the relative weight gain during the hydration:

$$S R = \frac{{w}_{h} - {w}_{d}}{{w}_{h}} \qquad ( 19 )$$

where Wₕ and W_{d} represent the mass of hydrated (swelled) and dried (xerogel) respectively.

### Compression Testing

Cylindrical hydrogel specimens (diameter: 10 mm, height: 4 mm) are prepared by 3D printing using ink G5 formulation containing the highest AS content. Uniaxial compression testing is conducted using a universal testing machine at a strain rate of 50 mm/min.

The ultimate compressive strength (UCS) is found to be_2700_kPa, and the Young's modulus is 2.1 kPa. The same specimen is subjected to 10 cycles of loading-unloading at 20% strain, showing no permanent set and full recovery of original shape, confirming the elastic nature of the network.

### Example 4—In Vitro Drug Release from Hybrid Hydrogel

Amoxicillin hydrochloride is incorporated at a concentration of 0.4-1.0 mg/mL into the hydrogel matrix prior to UV-curing. The printed hydrogel disc (diameter: 10 mm, thickness: 2 mm) is immersed in phosphate-buffered saline (PBS, pH 7.4) at 37° C.

Aliquots were collected at predetermined intervals up to 72 hours, and drug concentration was quantified using UV-vis spectroscopy at 330 nm. The cumulative release showed an initial release of 8-12% within the first hour, followed by a sustained release profile reaching 25% at 24 hours. The release kinetics fitted the Korsmeyer-Peppas model with a diffusion exponent of 0.4-0.7, indicating a non-Fickian diffusion mechanism.

No degradation of the drug was observed during the release period, as confirmed by the retention of the characteristic UV absorption peak and spectral overlay with the pristine drug.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

The term “shear-thinning behavior” refers to a non-Newtonian fluid characteristic where the viscosity of a material decreases with increasing shear rate. In the context of the present invention, this property enables the hydrogel ink to flow easily through a narrow nozzle during extrusion while maintaining sufficient viscosity at rest to prevent dripping or deformation of the printed structure. Shear-thinning is imparted primarily by cellulose nanocrystals (CNCs), which form a reversible physical network within the formulation, enhancing extrusion printability.

The term “yield stress” refers to the minimum stress required to initiate flow in a viscoplastic material. Below this threshold, the ink behaves like an elastic solid; above it, it flows like a viscous liquid. For the inventive hydrogel ink, the presence of CNCs establishes a yield stress that prevents unintentional dripping during non-extrusion intervals while allowing smooth extrusion when adequate pressure is applied. This ensures precise shape retention during layer-by-layer deposition.

The term “semi-interpenetrating polymer network (semi-IPN)” refers to a composite structure wherein a linear or branched polymer (in this case, amine-functionalized silicone elastomer, AS) is physically embedded within a chemically crosslinked network (formed from polymerized AM and HEMA monomers) without covalent bonding between them. The AS chains do not participate in the crosslinking reaction but remain entrapped within the hydrogel matrix, enhancing flexibility, oxygen permeability, and mechanical resilience while contributing to non-Fickian drug release behavior.

“Tan delta (tan δ)” is a dimensionless parameter defined as the ratio of the loss modulus (G″) to the storage modulus (G′) of a viscoelastic material. It represents the damping behavior or energy dissipation characteristics of the hydrogel. A tan δ value less than 0.1 indicates dominant elastic behavior with minimal energy loss under oscillatory shear, reflecting a highly crosslinked and stable network. The tan δ of the hydrogels in this invention is controlled via formulation parameters, particularly the ratio of AM to HEMA and AS content.

The phrase zero permanent deformation (also referred to as zero compression set) indicates that the hydrogel recovers its original shape completely after being subjected to compressive strain for a defined period and number of cycles. This property is characteristic of highly elastic materials and, in the context of the present invention, reflects the hybrid hydrogel's robust structural integrity. It is measured by subjecting the hydrogel to cyclic compression and observing its ability to return to its initial dimensions without residual strain.

“Non-Fickian anomalous transport behavior” refers to a diffusion process wherein the rate of drug release from the hydrogel is governed by both the diffusion of the drug molecules and the relaxation of the polymer matrix. In contrast to Fickian diffusion, where diffusion is solely concentration-driven, non-Fickian transport arises in systems like semi-IPNs where molecular mobility and matrix restructuring occur concurrently. The diffusion exponent (n) between 0.5 and 0.75, as derived from Korsmeyer-Peppas fitting, is indicative of this dual-controlled mechanism.

“Mesh size” refers to the average distance between crosslinking points in the polymer network, typically measured in ångströms (Å). It dictates the porosity of the hydrogel and influences both the diffusion rate of encapsulated drug molecules and the mechanical properties of the gel. In this invention, mesh size is modulated by varying the content of AS and the AM: HEMA ratio and is critical for achieving tunable drug release kinetics and oxygen permeability.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

References: The disclosures of the following references are incorporated by reference ADDIN EN.REFLIST
1. Oropallo, W. and L. A. Piegl, Ten challenges in 3D printing. Engineering with Computers, 2016. 32: p. 135-148.
2. Campbell, T., et al., Could 3D printing change the world. Technologies, Potential, and Implications of Additive Manufacturing, Atlantic Council, Washington, D C, 2011. 3.
3. Mora-Boza, A., et al., Glycerylphytate as an ionic crosslinker for 3D printing of multi-layered scaffolds with improved shape fidelity and biological features. Biomaterials science, 2020. 8(1): p. 506-516.
4. Urruela-Barrios, R., et al., Alginate/gelatin hydrogels reinforced with TiO2 and β-TCP fabricated by microextrusion-based printing for tissue regeneration. Polymers, 2019. 11(3): p. 457.
5. Hsu, K.-H., S. Gause, and A. Chauhan, Review of ophthalmic drug delivery by contact lenses. Journal of Drug Delivery Science and Technology, 2014. 24(2): p. 123-135.

What is claimed is:

1. An injectable, extrusion-printable, and biocompatible formulation for 3D printing of a drug-releasing hydrogel, comprising:
an aqueous dispersion of cellulose nanocrystals (CNCs) in an amount effective to impart shear-thinning behavior and yield stress sufficient to prevent nozzle dripping during pauses in extrusion;
a monomer mixture comprising acrylamide (AM) and 2-hydroxyethyl methacrylate (HEMA), wherein the AM to HEMA weight ratio is between 1:3 and 3:1;
a crosslinker;
a photoinitiator; and
an amine-functionalized silicone elastomer (AS);
wherein the formulation undergoes in situ ultraviolet-induced polymerization to form a semi-interpenetrating polymer network hydrogel with a mesh size of 50 Å to 150 Å, and
wherein the hydrogel exhibits a tan delta (tan δ) value of less than 0.1 under oscillatory shear, indicating high elasticity.

2. The injectable, extrusion-printable, and biocompatible formulation of claim 1, wherein the AS content is in a range of 0.1 wt % to 10 wt %, and the CNCs content is in a range of 5 wt % to 15 wt %, based on total formulation weight.

3. The injectable, extrusion-printable, and biocompatible formulation of claim 1, wherein the photoinitiator comprises lithium phenyl-2,4,6-trimethylbenzoylphosphinate (LAP), 2-hydroxy-4′-(2-hydroxyethoxy)-2-methylpropiophenone), 2,2′-Azobis [2-methyl-N-(2-hydroxyethyl) propionamide], or a combination thereof.

4. The injectable, extrusion-printable, and biocompatible formulation of claim 1, wherein the crosslinker comprises N,N′-methylenebisacrylamide (MBA), water-soluble diacrylates, or a combination thereof.

5. The injectable, extrusion-printable, and biocompatible formulation of claim 1, wherein the formulation exhibits a shear-thinning behavior, characterized by a decrease in viscosity from 10⁴ Pa·s at a shear rate of 0.1 s⁻¹ to 0.1 Pa·s at a shear rate of 100 s⁻¹.

6. The injectable, extrusion-printable, and biocompatible formulation of claim 1, wherein the formulation displays a yield stress of approximately 1.8 Pa to 2.7 Pa when tested in compression mode.

7. The injectable, extrusion-printable, and biocompatible formulation of claim 1, wherein the semi-interpenetrating polymer network hydrogel exhibits zero permanent deformation following cyclic compression loading, a diffusion exponent (n) in the range of 0.5 to 0.75 indicative of non-Fickian anomalous transport behavior, and tunable oxygen permeability and mesh size modulated by the content of the AS.

8. The injectable, extrusion-printable, and biocompatible formulation of claim 1, wherein the semi-interpenetrating polymer network hydrogel has a Young's modulus greater than 10 kPa and an ultimate compressive strength of at least 50 kPa.

9. The injectable, extrusion-printable, and biocompatible formulation of claim 1, wherein the semi-interpenetrating polymer network hydrogel maintains dimensional and structural stability after immersion in phosphate-buffered saline at 37° C. for at least 10 days, and wherein the drug encapsulated therein retains at least 90% of its original chemical integrity and ultraviolet absorbance profile after storage for at least one month.

10. The injectable, extrusion-printable, and biocompatible formulation of claim 1, wherein the semi-interpenetrating polymer network hydrogel adheres to the periphery of a contact lens without delamination, distortion, or optical interference, and wherein the semi-interpenetrating polymer network hydrogel conforms to the curvature of the cornea or sclera under ambient conditions.

11. A method of fabricating a structurally stable semi-interpenetrating polymer network hydrogel, comprising:
dispersing cellulose nanocrystals (CNCs) in water to form a shear-thinning base fluid;
mixing the shear-thinning base fluid with a monomer mixture, a crosslinker, a photoinitiator, and an amine-functionalized silicone elastomer to form a precursor ink complex;
extruding the precursor ink complex into a predefined pattern using a microextrusion-based 3D printer, and subjecting extruded predefined pattern to continuous ultraviolet irradiation at a wavelength of 365 nm for a duration of 5 to 10 seconds to initiate polymerization and form the structurally stable semi-interpenetrating polymer network hydrogel,
wherein simultaneous ultraviolet exposure and extrusion are employed to achieve in situ gelation during layer-by-layer deposition.

12. The method of claim 11, further comprising incorporating a therapeutic agent into the precursor ink complex, and the therapeutic agent is selected from antibiotics, anti-inflammatory drugs, anti-glaucoma drugs, or a combination thereof.

13. The method of claim 11, wherein the precursor ink complex comprises a monomer mixture of acrylamide (AM) and 2-hydroxyethyl methacrylate (HEMA), wherein the weight ratio of AM to HEMA is between 1:3 and 3:1, an amine-functionalized silicone elastomer (AS) in an amount of 0.1 wt % to 10 wt %, and the CNCs in an amount of 5 wt % to 15 wt %, each based on the total weight of the precursor ink complex.

14. The method of claim 11, wherein the structurally stable semi-interpenetrating polymer network hydrogel sustains drug release for at least 8 hours post-application.

15. The method of claim 11, wherein the structurally stable semi-interpenetrating polymer network hydrogel exhibits no permanent deformation after undergoing at least 10 cycles of 20% strain.

16. The method of claim 11, wherein the structurally stable semi-interpenetrating polymer network hydrogel is printed in annular patterns with diameters of at least 10 mm.

17. A drug delivery system comprising the semi-interpenetrating polymer network hydrogel of claim 1 printed on a substrate.

18. The drug delivery system of claim 17, wherein the substrate comprises contact lenses, ocular patches, corneal bandages, or biodegradable ocular films.

19. The drug delivery system of claim 17, wherein the semi-interpenetrating polymer network hydrogel has an oxygen permeability that exceeds that of poly (HEMA)-based hydrogel lenses by at least 25%.

20. The drug delivery system of claim 17, wherein the semi-interpenetrating polymer network hydrogel is printed in a manner that allows the hydrogel to be detached and re-adhered without delaminating or compromising its structural integrity.
